(19) United States
(12) Patent Application Publication
(10) Pub. No.: US 2024/0189329 A1
(43) Pub. Date: Jun. 13, 2024

(54) WATER-SOLUBLE CANNABIS CANNABINOID SYSTEMS FOR INFUSING PRODUCTS WITH NANOEMULSIONS HAVING NANOSCALE SIZES

(71) Applicant: HEXO OPERATIONS INC., GATINEAU (CA)

(72) Inventor: Jamie Savard, Gatineau (CA)

(21) Appl. No.: 18/287,769

(22) PCT Filed: Apr. 26, 2022
(86) PCT No.: PCT/CA2022/050634

§ 371 Date: Oct. 20, 2023

Related U.S. Application Data
(60) Provisional application No. 63/179,988, filed on Apr. 26, 2021.

(51) Int. Cl. A61K 31/00; A23L 2/52; A61K 9/107; A61K 47/10; A61K 47/14; A61K 47/18; A61K 47/26

(52) U.S. Cl. CPC A61K 31/658; A23L 2/52; A61K 9/1075; A61K 47/10; A61K 47/14; A61K 47/183; A61K 47/26

(57) ABSTRACT

The present disclosure addresses the need to obtain cannabinoid emulsification systems that are capable of generating controllable, stable small particle size for making cannabis-infused products. The disclosure relates to self-emulsifying cannabinoid systems comprise a water phase comprising a polyhydric alcohol and a monohydric alcohol, and an oil phase comprising an emulsifier and a cannabinoid component, the cannabinoid component including a cannabinoid. The water phase and oil phase combine to generate the nanoemulsion.

### CROSS-REFERENCE TO RELATED APPLICATION

This application claims priority from U.S. Provisional Patent Application 63/179,988 filed on Apr. 26, 2021, and incorporated by reference herein.

### TECHNICAL FIELD

The present disclosure relates generally to the field of cannabinoid systems, and more particularly to cannabinoid systems for infusing products such as beverages, topicals and foodstuffs.

### BACKGROUND

Cannabis is a genus of flowering plant in the family Cannabaceae. The number of species within the genus is disputed. Three species may be recognized, Cannabis sativa, Cannabis indica and Cannabis ruderalis. C. ruderalis may be included within C. sativa; or all three may be treated as subspecies of a single species, C. sativa. The genus is indigenous to central Asia and the Indian subcontinent. As the cannabis industry grows and an increasing number of new products are available for consumers, it has become paramount to produce products that are safe and well controlled in experience for the consumer. One of the main aspects of this being a predictable fast-onset of experience. Present cannabis-infused (such as edibles, topicals and non-edible liquids) products are often criticized as having significant unpredictability in terms of on-set, with a marked disparity of up to 2 hours between individuals who have consumed the same product and quantity.

Consumers often do not understand this aspect of cannabis-infused products use and may consume a greater than intended amount of cannabis-infused product before the cannabis active molecules contained therein have taken effect, often resulting in profoundly adverse effects. Although ample experimental evidence demonstrates that cannabis is not particularly lethal and, to date, no deaths have been directly attributed to the acute physical toxicity of cannabis, episodes of severe cannabis-induced behavioral impairment are common, and can result in cognitive and motor impairment, extreme sedation, agitation, anxiety, cardiac stress, and/or vomiting.

Moreover, the amount of cannabis active molecules in cannabis-infused products can vary across a single product and across batches formulated at different times, making it difficult for users to estimate how much cannabis active molecules they consume. The lack of consistency and the delayed intoxication has also been reported with use of other cannabis-infused products containing various cannabis active molecule profiles and may cause both new and experienced users of cannabis to consume higher than intended amounts of the cannabis active molecules.

The nascent cannabis industry thus faces significant challenges in view of such problems and risks having consumers demand alternative solutions perceived as being less risky, which could have significant commercial impacts.

### SUMMARY

This Summary is provided to introduce a selection of concepts in a simplified form that are further described below in the Detailed Description. This Summary is not intended to identify key aspects or essential aspects of the claimed subject matter.

In one broad aspect, the present disclosure relates to a self-emulsifying cannabinoid system comprising a cannabinoid component including a cannabinoid, and an emulsifier, for use with a water phase comprising a polyhydric alcohol and a monohydric alcohol to generate a nanoemulsion.

In one broad aspect, the present disclosure relates to a self-emulsification cannabinoid system comprising a water phase comprising a polyhydric alcohol and a monohydric alcohol, and an oil phase comprising an emulsifier and a cannabinoid component, the cannabinoid component including a cannabinoid, wherein the water phase and oil phase combine to generate a nanoemulsion.

In one broad aspect, the present disclosure relates to a nanoemulsion comprising a cannabinoid component including a cannabinoid, an emulsifier, a polyhydric alcohol and a monohydric alcohol.

In specific implementations, the self-emulsification cannabinoid system and/or the nanoemulsion further includes one or more of the following:
the polyhydric alcohol is one or more of glycerol, arabitol, Hydrogenated starch hydrolysates (HSHs), lactitol, mannitol, maltitol, sorbitol, xylitol, erythritol, and isomalt, preferably glycerol, sorbitol, or a combination thereof, more preferably the polyhydric alcohol is glycerol.
the polyhydric alcohol is glycerol.
the nanoemulsion comprises the polyhydric alcohol in an amount of from about 10 wt. % to about 20 wt. %, preferably from about 12.5 wt. % to about 18.5 wt. %, more preferably from about 14.5 wt. % to about 16.5 wt. %, or more preferably about 15 wt. %.
the nanoemulsion comprises the monohydric alcohol in an amount of from about 1.0 wt. % to about 10 wt. %, preferably from about 2.0 wt. % to about 8.0 wt. %, more preferably from about 2.0 to about 5.5 wt. %.
the monohydric alcohol includes methanol, ethanol, propan-1-ol, propan-2-ol, butan-1-ol, butan-2-ol, 2-methyl propan-1-ol, or 2-methyl propan-1-ol, preferably ethanol.
the monohydric alcohol is ethanol.
the cannabinoid component further comprises a carrier oil.
the carrier oil is selected from borage oil, coconut oil, cottonseed oil, soybean oil, safflower oil, sunflower oil, castor oil, corn oil, olive oil, palm oil, peanut oil, almond oil, sesame oil, rapeseed oil, peppermint oil, poppy seed oil, canola oil, palm kernel oil, hydrogenated soybean oil, hydrogenated vegetable oils, glyceryl esters of saturated fatty acids, glyceryl behenate, glyceryl distearate, glyceryl isostearate, glyceryl laurate, glyceryl monooleate, glyceryl, monolinoleate, glyceryl palmitate, glyceryl palmitostearate, glyceryl ricinoleate, glyceryl stearate, polyglyceryl 10-oleate, polyglyceryl 3-oleate, polyglyceryl 4-oleate, polyglyceryl 10-tetralinoleate, behenic acid, medium-chain triglycerides (MCT), long chain triglycerides (LCT), and any combination thereof.
the carrier oil is MCT.
the cannabinoid and the carrier oil are present in a ratio by weight of from about 0.40 to about 1.00, preferably from about 0.45 to about 0.80, more preferably from about 0.50 to about 0.75, even more preferably from about 0.56 to about 0.71.
the emulsifier has a Hydrophilic-lipophilic balance (HLB) 10.
the emulsifier includes a non-ionic emulsifier.
the non-ionic emulsifier includes polyoxyethylene monostearate, polyoxyethylene monooleate, polyoxyethylene sorbitan monolaurate, polyoxyethylene sorbitan monolaurate, polyoxyethylene sorbitan monopalmitate, polyoxyethylene sorbitan monostearate, polyoxyethylene sorbitan monostearate, polyoxyethylene sorbitan tristearate, polyoxyethylene sorbitan monooleate, polyoxyethylene sorbitan monooleate, polyoxyethylene sorbitan trioleate, polyoxyethylene-(15)-stearic acid, polyoxyethylene-(20)-stearyl alcohol, polyoxyethylene-(23)-lauryl alcohol, PEG-40 hydrogenated castor oil, PEG-35 castor oil, Solutol HS-15, or any combinations thereof, more preferably the emulsifier includes a non-ionic polysorbate surfactant, even more preferably the emulsifier includes polysorbate 80.
the cannabinoid includes THC, CBD, CBG, CBN, or any combinations thereof.
the nanoemulsion comprises a cannabinoid content of about 0.1 mg/ml or more.
the nanoemulsion has a particle size of about 120 nm or less, preferably of about 100 nm or less, more preferably of about 80 nm or less, even more preferably of about 50 nm or less, even yet more preferably of about 35 nm or less, even more particularly of from about 25 nm to about 35 nm.

The present disclosure also relates to a cannabis-infused product comprising the cannabinoid self-emulsification system or the nanoemulsion.

In one broad aspect, the present disclosure relates to a process for manufacturing a cannabinoid nanoemulsion, comprising: providing a water phase comprising a polyhydric alcohol and a monohydric alcohol; providing an oil phase comprising an emulsifier and a cannabinoid component, the cannabinoid component including a cannabinoid; and adding the oil phase to the water phase in a stepwise manner while mixing to generate the cannabinoid nanoemulsion.

In specific implementations, the process further includes one or more of the following:
the stepwise manner comprises adding the oil phase dropwise to the water phase.
the mixing is performed with stirring at about 800 rpm or less, preferably at about 600 rpm or less.
the mixing is performed at a temperature of between about 45° C. and about 55° C.
further comprising adjusting a pH of the nanoemulsion to about 6.5 or less, preferably to between about 2.5 and about 4.5, more preferably between about 3.8 and about 4.3.
wherein adjusting a pH comprises adding ascorbic acid for adjusting the pH.
wherein the water phase is free from ascorbic acid prior to mixing with the oil phase.
the polyhydric alcohol is one or more of glycerol, arabitol, Hydrogenated starch hydrolysates (HSHs), lactitol, mannitol, maltitol, sorbitol, xylitol, erythritol, and isomalt, preferably glycerol, sorbitol, or a combination thereof, more preferably the polyhydric alcohol is glycerol.
the nanoemulsion comprises the polyhydric alcohol in an amount of from about 10 wt. % to about 20 wt. %, preferably from about 12.5 wt. % to about 18.5 wt. %, more preferably from about 14.5 wt. % to about 16.5 wt. %, or more preferably about 15 wt. %.
the nanoemulsion comprises the monohydric alcohol in an amount of from about 1.0 wt. % to about 10 wt. %, preferably from about 2.0 wt. % to about 8.0 wt. %, more preferably from about 2.0 to about 5.5 wt. %.
the monohydric alcohol includes methanol, ethanol, propan-1-ol, propan-2-ol, butan-1-ol, butan-2-ol, 2-methyl propan-1-ol, or 2-methyl propan-1-ol, preferably ethanol.
the monohydric alcohol is ethanol.
the cannabinoid component further comprises a carrier oil.
the carrier oil is selected from borage oil, coconut oil, cottonseed oil, soybean oil, safflower oil, sunflower oil, castor oil, corn oil, olive oil, palm oil, peanut oil, almond oil, sesame oil, rapeseed oil, peppermint oil, poppy seed oil, canola oil, palm kernel oil, hydrogenated soybean oil, hydrogenated vegetable oils, glyceryl esters of saturated fatty acids, glyceryl behenate, glyceryl distearate, glyceryl isostearate, glyceryl laurate, glyceryl monooleate, glyceryl, monolinoleate, glyceryl palmitate, glyceryl palmitostearate, glyceryl ricinoleate, glyceryl stearate, polyglyceryl 10-oleate, polyglyceryl 3-oleate, polyglyceryl 4-oleate, polyglyceryl 10-tetralinoleate, behenic acid, medium-chain triglycerides (MCT), long chain triglycerides (LCT), and any combination thereof.
the carrier oil is MCT.
the cannabinoid and the carrier oil are present in a ratio by weight of from about 0.40 to about 1.00, preferably from about 0.45 to about 0.80, more preferably from about 0.50 to about 0.75, even more preferably from about 0.56 to about 0.71.
the surfactant has a Hydrophilic-lipophilic balance (HLB) 10.
the emulsifier includes a non-ionic emulsifier.
the non-ionic emulsifier includes polyoxyethylene monostearate, polyoxyethylene monooleate, polyoxyethylene sorbitan monolaurate, polyoxyethylene sorbitan monolaurate, polyoxyethylene sorbitan monopalmitate, polyoxyethylene sorbitan monostearate, polyoxyethylene sorbitan monostearate, polyoxyethylene sorbitan tristearate, polyoxyethylene sorbitan monooleate, polyoxyethylene sorbitan monooleate, polyoxyethylene sorbitan trioleate, polyoxyethylene-(15)-stearic acid, polyoxyethylene-(20)-stearyl alcohol, polyoxyethylene-(23)-lauryl alcohol, PEG-40 hydrogenated castor oil, PEG-35 castor oil, Solutol HS-15, or any combinations thereof, more preferably the emulsifier includes a non-ionic polysorbate surfactant, even more preferably the emulsifier includes polysorbate 80.
the cannabinoid includes THC, CBD, CBG, CBN, or any combinations thereof.
the nanoemulsion comprises a cannabinoid content of about 0.1 mg/ml or more.
the nanoemulsion has a particle size of about 120 nm or less, preferably of about 100 nm or less, more preferably of about 80 nm or less, even more preferably of about 50 nm or less, even yet more preferably of about 35 nm or less, even more particularly of from about 25 nm to about 35 nm.

Other aspects and features of the cannabinoid systems, methods and cannabis-infused products of the present disclosure will become apparent to those ordinarily skilled in the art upon review of the following description of specific embodiments.

### DETAILED DESCRIPTION

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the suitable methods and materials are described below.

The present inventors have surprisingly and unexpectedly discovered that at least some of the problems discussed above with respect to cannabis-infused products may be addressed by providing cannabis-infused products which have an onset of a cannabis-associated effect in a manner which can be more consistent and controlled. Advantageously, use of the herein described cannabis-infused products may provide a cannabis-associated effect that can be perceived by the user as being relatively faster compared to cannabis-infused products that do not include the cannabinoid systems from the present disclosure. In order to achieve this, the present specification discloses cannabis-infused products, which are designed to provide a fast onset of the cannabinoids contained therein and which are stable over time, thus providing clear commercial advantages to the cannabis industry.

Advantageously, such cannabis-infused product may afford an enhanced and more consistent user experience—e.g., one can substantially tailor his/her cannabis user experience by consuming such cannabis-infused product.

In the present specification, the expression “cannabis-infused” will be used with reference to consumer products (such as a edibles, topical solids and non-edible liquids), which contain a cannabis active molecule (such as one or more cannabinoid) that has been admixed or infused with other ingredients forming the consumer product. For example, in the case of a cannabis-infused beverage product, this beverage product can be made by infusing the herein described cannabinoid system in a beverage base, preferably a cannabinoid-less beverage base.

The infusion can be performed by mixing a powdered form of the cannabinoid system and/or a liquid form of the cannabinoid system with the beverage base.

There are a number of options to obtain the herein described cannabis-infused product.

For example, one can design a cannabis-infused product containing a cannabinoid system in accordance with the present disclosure, where the cannabinoid system affords an increased bioavailability of the cannabinoid contained therein.

For example, one can design a precursor cannabinoid system, which includes a cannabinoid system or part thereof in accordance with the present disclosure, and then infuse a product base with the precursor cannabinoid system or part thereof to obtain the cannabis-infused product.

These and other examples of implementation of the present disclosure will become apparent to the person of skill in view of the disclosure as a whole.

### Cannabinoid Systems

The cannabinoid systems of the present disclosure are generally self-emulsifying cannabinoid systems.

As used herein, the term “cannabinoid systems” is used to refer to the combination of elements or a subpart thereof that when processed according to the teachings described herein generate a nanoemulsion. As such, the cannabinoid systems when completely processed are in the form of the nanoemulsion and as such, depending on the context which is readily understandable to the person of skill, the term “cannabinoid systems” may also refer to the generated nanoemulsion.

In one broad aspect, the cannabinoid systems can be used for making a cannabis-infused product. In some embodiments, the cannabinoid systems can be used by its manufacturer to obtain the cannabis-infused product, ready for packaging and commercialization. In some embodiments, the cannabinoid systems or some elements thereof may be packaged and sold to a product manufacturer, which can then use same to obtain the cannabis-infused product. In some embodiments, the cannabinoid systems may be packaged and commercialized alone or together with a base product such that the end-user may use same to obtain the cannabis-infused product.

As used herein, the term “product base” refers to dry, semi-dry or liquid composition, which is used to obtain the herein described cannabis-infused product. For example, the product base may be a finished product to which the cannabinoid composition is added to (e.g., sprinkled upon, added to, incorporated into, etc.) in order to obtain the cannabis-infused product. Alternatively, the product base may be an ingredient or a mixture of ingredients for making the cannabis-infused product. In such embodiment, the cannabinoid systems can be mixed with the product base to obtain a blend, which can then be used for manufacturing the cannabis-infused product by further processing (e.g., baking, cooking, extruding, pressing, homogenizing, etc.).

As used herein, the term “cannabis-infused product” refers to commercial solid, semi-solid, or liquid products containing a cannabinoid for which it is desirable to obtain a fast onset. For example, cannabis-infused products such as edibles, topical solids and non-edible liquids. For example, beverages (e.g., alcoholic, non-alcoholic, juices, sparkling water, etc.), liquid products (oral sprays, drops, liquid-filled capsules, etc.), human solid edibles (e.g., baked goods, cereals, etc.), pet edibles (e.g., pet food, pet chew, etc.), cosmetics (balms, topicals, etc.), and/or confections (e.g., lozenges, chewing gum, mints, chocolates, candies, gummies, etc.).

As used herein, the term “self-emulsifying” refers to a system that includes elements that when combined, under low energy conditions (such as gentle agitation), spontaneously generates a nanoemulsion. Advantageously, the nanoemulsions formed through the self-emulsifying systems described herein have low particle sizes and as such, additional processing of the nanoemulsions through a high shear step (such as microfluidization) to achieve low particle size is unnecessary. In other words, the manufacturing process is a low energy process. This in turn may reduce manufacturing costs, increase productivity and streamline the manufacturing process.

In some embodiments, the self-emulsifying cannabinoid systems comprise a water phase comprising a polyhydric alcohol and a monohydric alcohol, and an oil phase comprising an emulsifier and a cannabinoid component, the cannabinoid component including a cannabinoid. The water phase and oil phase combine to generate the nanoemulsion.

As used herein, the term “nanoemulsion” means an emulsion which is mainly constituted of particles having a particle size distribution which is less than about 1000 nm. In other words, the emulsion is made of at least 95%, or at least 96%, or at least 97%, or at least 98%, or at least 99% of particles in the nanometric range (i.e., from 0 to 1000 nm). Preferably, the nanoemulsion described herein has a particle size of about 120 nm or less. For example, the nanoemulsion has a particle size of about 100 nm or less, more preferably of about 80 nm or less, even more preferably of about 50 nm or less, even yet more preferably of about 35 nm or less, even more particularly of from about 25 nm to about 35 nm.

The term “particle size”, as used herein, refers to a volume based particle size measured, for example, by dynamic light scattering (DLS) which is a non-invasive, well-established technique for measuring the size and size distribution of molecules and particles typically in the submicron region. The DLS instrument has a laser light source which is used to illuminate the particles. Then, the generated scattered light fluctuates at a speed depending on the particle size. With the velocity of the Brownian motion, derived from the analysis of these fluctuations, and the Stokes-Einstein equation, particle size can be obtained. The particle size is reported as a volume equivalent sphere diameter.

Advantageously, the herein described cannabinoid systems are highly soluble in water or aqueous composition. Thus, the present disclosure provides convenient cannabinoid systems that may be readily used in the preparation of compositions that contain an aqueous composition component, such as topicals, beverages, foodstuffs, and the like.

The cannabinoid systems of the present disclosure show high emulsion stability evidenced, for example, by the clarity of cannabinoid systems and resultant cannabis-infused products (such as beverages), as well as the stability of the products and cannabinoids therein.

The cannabinoid systems of the present disclosure, and in particular the cannabinoid systems comprising a polyhydric alcohol and a monohydric alcohol, are suitable for addition to foodstuffs and beverages. In particular, the cannabinoid systems of the present disclosure are capable of preparing beverages that are shelf stable for extended periods of time (e.g., for 3 months at accelerated stability at 40° C., which is roughly representative of a 1-year stability at room temperature). Thus, the cannabinoid systems of the present disclosure are capable of providing cannabinoid stability in beverages, which is key for commercial products that require extended shelf-life.

The cannabinoid systems of the present disclosure, and in particular the cannabinoid systems comprising a polyhydric alcohol and a monohydric alcohol, are also advantageous in preparing transparent, translucent and/or clear beverages over a broad range of different flavour, colour and ionic conditions. For example, cannabinoid systems of the present disclosure comprising both polyhydric alcohol and a monohydric alcohol can exhibit a turbidity of less than 5.0 Nephelometric Turbidity Units (NTU) as measured with a nephelometer. For example, a turbidity of no more than 4 NTU, or no more than 3.0 NTU, or no more than 2.0 NTU, or no more than 1.0 NTU.

The cannabinoid systems of the present disclosure are also advantageous in respect of their preparation. For example, traditional methods of emulsification make use of high shear homogenizers or sonication-based methods that are unable to produce highly uniform particle sizes leading to cloudy, unstable emulsions. Further, while spontaneous formation of cannabinoid containing microemulsions is reported in the literature, these previous methods necessarily used very high concentrations of artificial emulsifiers and were not always fully dilutable, leading to soapy, cloudy, expensive and unpalatable cannabinoid systems. Additionally, water-soluble cannabinoids may also be produced by molecular encapsulation (i.e., in cyclodextrins and modified starches), but these cannabinoid systems are usually cloudy and use unnatural ingredients. The presently disclosed cannabinoid systems are generally capable of avoiding such cannabinoid system challenges.

Accordingly, the present disclosure provides convenient cannabinoid systems of cannabinoids that may be readily used in the preparation of compositions that contain an aqueous composition component, such as topicals, beverages and foodstuffs, which are capable of producing cannabis-infused products that are transparent, translucent and/or clear and that are shelf stable for extended periods of time.

Individually and separately, these exemplary improvements produce advantageous cannabinoid systems and dosage forms, and, at times, the combinations of ingredients can provide beneficial effects on preparation, storage, distribution and/or end use of the cannabinoid systems. Further improvements are described herein or will become evident from the present disclosure.

The cannabinoid systems described herein serve as a convenient water-soluble dosage form of cannabinoids for use in beverages, topicals and foods. The formations are suitably in a nontoxic consumable liquid or solid form. For example, cannabinoid systems described herein can be made using food grade excipients. Suitably, the cannabinoid systems provide stability, solubility in water, have minimal flavour and odor, are calorie-free, and are natural in origin.

In a particular embodiment, the cannabinoid systems described herein are transparent and/or clear.

As used herein, “water-soluble” has its ordinary meaning in referring to the ability of a cannabinoid system or component thereof to dissolve when the object is placed in water or an aqueous composition. For example, when the object is mixed with water at room temperature or slightly above (e.g., about 25° C. to about 50° C.).

As used herein, “transparent” has its ordinary meaning of having the property of allowing light to pass through without appreciable scattering. Transparency may be measured by a transparency meter (also called a clarity meter) and is identified by an object's total transmittance, which is the ratio of transmitted light to the incident light. In an embodiment herein, transparent means a total transmittance of between about 80% and 100%. In an embodiment, transparent means a total transmittance of about 90%, about 91%, about 92%, about 93%, about 94%, about 95%, about 96%, about 97%, about 98%, or about 99%. In an embodiment, transparent means a total transmittance of at least 94%. Visually, the skilled person will appreciate that an object is transparent if it is easy to see through without significant distortion.

Transparency may be equated with clarity (e.g., “clear” or “substantially clear”). As used herein, the term “substantially clear” means that the visible turbidity or cloudiness is very slight (e.g., barely visible to the naked-eye). Turbidity or cloudiness may be measured by a number of means known to one of ordinary skill in the art, including by refractometry, nephelometry, chromatography or spectrometry. In an embodiment, turbidity may be measured by a Nephelometer to determine the Nephelometric Turbidity Units (NTU). In an embodiment, “substantially clear” means that the liquid has an NTU of less than 50. In an embodiment, “substantially clear” means that the liquid has an NTU of ≤25, ≤20, ≤15 or ≤10.

As used herein, the term “clear” means that there is no visible turbidity or cloudiness to the naked-eye. In an embodiment, “clear” means that the liquid has an NTU of ≤5. In an embodiment, “clear” means that the liquid has an NTU of about 5, about 4, about 3, about 2, about 1 or less (e.g., close to or at zero).

As opposed to transparent objects and liquids which generally appear clear, as used herein the term “translucent” means that the objects permit the passage of light but does not appear clear. Translucent objects typically diffuse light such that objects cannot be observed clearly on the opposite side. “Translucent” is defined by either transmittance or reflectance measurement modes (see HunterLab definition, which is available at HunterLab website, on the translucent beverage color measurement page).

In an embodiment, the cannabinoid systems of the present disclosure are a powder. The powder may, for example, be prepared by drying liquid cannabinoid systems of the present disclosure.

### Cannabinoid Component Includes a Cannabinoid

The cannabinoid systems of the present disclosure comprise a “cannabinoid component” that includes a cannabinoid.

As used herein, the term “cannabinoid” generally refers to any chemical compound that acts upon a cannabinoid receptor such as CB1 and CB2. Examples of cannabinoids include, but are not limited to, cannabichromanon (CBCN), cannabichromene (CBC), cannabichromevarin (CBCV), cannabicitran (CBT), cannabicyclol (CBL), cannabicyclovarin (CBLV), cannabidiol (CBD, defined below), cannabidiolic acid (CBDA), cannabidiol monomethylether (CBDM), cannabidiol-C4 (CBD-C4), cannabidiorcol (CBD-C1), cannabidiphorol (CBDP), cannabidivarin (CBDV), cannabielsoin (CBE), cannabifuran (CBF), cannabigerol (CBG), cannabigerol monomethylether (CBGM), cannabigerolic acid (CBGA), cannabigerovarin (CBGV), cannabinodiol (CBND), cannabinodivarin (CBVD), cannabinol (CBN), cannabinol methylether (CBNM), cannabinol propyl variant (CBNV), cannabinol-C2 (CBN-C2), cannabinol-C4 (CBN-C4), cannabiorcol (CBN-C1), cannabiripsol (CBR), cannabitriol (CBO), cannabitriolvarin (CBTV), cannabivarin (CBV), dehydrocannabifuran (DCBF), Δ7-cis-iso tetrahydrocannabivarin, tetrahydrocannabinol (THC, defined below), Δ9-tetrahydrocannabinolic acid (THC-A) including either or both isomers 2-COOH-THC (THCA-A) and 4-COOH-THC (THCA-B), Δ9-tetrahydrocannabiorcol (THC-C1), tetrahydrocannabivarinic acid (THCVA), tetrahydrocannabivarin (THCV), ethoxy-cannabitriolvarin (CBTVE), trihydroxy-Δ9-tetrahydrocannabinol (triOH-THC), 10-ethoxy-9hydroxy-Δ6a-tetrahydrocannabinol, 8,9-dihydroxy-Δ6a-tetrahydrocannabinol, 10-oxo-Δ6a-tetrahydrocannabionol (OTHC), 3,4,5,6-tetrahydro-7-hydroxy-α-α-2-trimethyl-9-n-propyl-2, 6-methano-2H-1-benzoxocin-5-methanol (OH-iso-HHCV), Δ6a,10a-tetrahydrocannabinol (Δ6a,10a-THC), Δ8-tetrahydrocannabivarin (Δ8-THCV), Δ9-tetrahydrocannabiphorol (Δ9-THCP), Δ9-tetrahydrocannabutol (Δ9-THCB), derivatives of any thereof, and combinations thereof. Further examples of suitable cannabinoids are discussed in at least WO2017/190249 and U.S. Patent Application Pub. No. US2014/0271940, which are each incorporated by reference herein in their entirety.

Cannabidiol (CBD) means one or more of the following compounds: Δ2-cannabidiol, Δ5-cannabidiol (2-(6-isopropenyl-3-methyl-5-cyclohexen-1-yl)-5-pentyl-1,3-benzenediol); Δ4-cannabidiol (2-(6-isopropenyl-3-methyl-4-cyclohexen-1-yl)-5-pentyl-1,3-benzenediol); Δ3-cannabidiol (2-(6-isopropenyl-3-methyl-3-cyclohexen-1-yl)-5-pentyl-1,3-benzenediol); Δ3,7-cannabidiol (2-(6-isopropenyl-3-methylenecyclohex-1-yl)-5-pentyl-1,3-benzenediol); Δ2-cannabidiol (2-(6-isopropenyl-3-methyl-2-cyclohexen-1-yl)-5-pentyl-1,3-benzenediol); Δ1-cannabidiol (2-(6-isopropenyl-3-methyl-1-cyclohexen-1-yl)-5-pentyl-1,3-benzenediol); and Δ6-cannabidiol (2-(6-isopropenyl-3-methyl-6-cyclohexen-1-yl)-5-pentyl-1,3-benzenediol). In a preferred embodiment, and unless otherwise stated, CBD means Δ2-cannabidiol.

Tetrahydrocannabinol (THC) means one or more of the following compounds: Δ8-tetrahydrocannabinol (Δ8-THC), Δ8-tetrahydrocannabivarin (Δ8-THCV), Δ9-cis-tetrahydrocannabinol (cis-THC), Δ9-tetrahydrocannabinol (Δ9-THC), Δ10-tetrahydrocannabinol (Δ10-THC), Δ9-tetrahydrocannabinol-C4 (THC-C4), Δ9-tetrahydrocannabinolic acid-C4 (THCA-C4), synhexyl (n-hexyl-Δ3THC). In a preferred embodiment, and unless otherwise stated, THC means one or more of the following compounds: Δ9-tetrahydrocannabinol and Δ8-tetrahydrocannabinol.

Synthetic cannabinoids and semisynthetic cannabinoids encompass a variety of distinct chemical classes, for example and without limitation: the classical cannabinoids structurally related to THC, the non-classical cannabinoids (cannabimimetics) including the aminoalkylindoles, 1,5-diarylpyrazoles, quinolines, and arylsulfonamides as well as eicosanoids related to endocannabinoids.

In many cases, a cannabinoid can be identified because its chemical name will include the text string “*cannabi*”. However, there are a number of cannabinoids that do not use this nomenclature, such as for example those described herein.

Within the context of this disclosure, where reference is made to a particular cannabinoid, each of the acid and/or decarboxylated forms are contemplated as both single molecules and mixtures. In addition, salts of cannabinoids are also encompassed, such as salts of cannabinoid carboxylic acids.

As well, any and all isomeric, enantiomeric, or optically active derivatives are also encompassed. In particular, where appropriate, reference to a particular cannabinoid includes both the “A Form” and the “B Form”. For example, it is known that THCA has two isomers, THCA-A in which the carboxylic acid group is in the 1 position between the hydroxyl group and the carbon chain (A Form) and THCA-B in which the carboxylic acid group is in the 3 position following the carbon chain (B Form).

In an embodiment, the cannabinoid is a cannabinoid dimer. The cannabinoid may be a dimer of the same cannabinoid (e.g., THC-THC) or different cannabinoids. In an embodiment, the cannabinoid may be a dimer of THC, including for example cannabisol.

In an embodiment, the cannabinoid is THC, CBD, CBG, CBN, or any combinations thereof, in similar or different amounts.

In an embodiment, the cannabinoid is THC.

In an embodiment, the cannabinoid is CBD.

As is known in the art, various cannabinoids can be used in combination to achieve a desired effect in a user. Certain specific ratios of cannabinoids may be useful to produce the feeling of physical and/or emotional satisfaction and/or may be useful in the treatment or management of specific diseases or conditions.

In some embodiments, the cannabinoid component includes a mixture of THC and CBD.

The w/w ratio of THC to CBD in the cannabinoid component may be between about 1:1000 and about 1000:1. Preferably, the (w/w) ratio of THC to CBD in the component may be about 1:1000, about 1:900, about 1:800, about 1:700, about 1:600, about 1:500, about 1:400, about 1:300, about 1:250, about 1:200, about 1:150, about 1:100, about 1:90, about 1:80, about 1:70, about 1:60, about 1:50, about 1:45, about 1:40, about 1:35, about 1:30, about 1:29, about 1:28, about 1:27, about 1:26, about 1:25, about 1:24, about 1:23, about 1:22, about 1:21, about 1:20, about 1:19, about 1:18, about 1:17, about 1:16, about 1:15, about 1:14, about 1:13, about 1:12, about 1:11, about 1:10, about 1:9, about 1:8, about 1:7, about 1:6, about 1:5, about 1:4.5, about 1:4, about 1:3.5, about 1:3, about 1:2.9, about 1:2.8, about 1:2.7, about 1:2.6, about 1:2.5, about 1:2.4, about 1:2.3, about 1:2.2, about 1:2.1, about 1:2, about 1:1.9, about 1:1.8, about 1:1.7, about 1:1.6, about 1:1.5, about 1:1.4, about 1:1.3, about 1:1.2, about 1:1.1, about 1:1, about 1.1:1, about 1.2:1, about 1.3:1, about 1.4:1, about 1.5:1, about 1.6:1, about 1.7:1, about 1.8:1, about 1.9:1, about 2:1, about 2.1:1, about 2.2:1, about 2.3:1, about 2.4:1, about 2.5:1, about 2.6:1, about 2.7:1, about 2.8:1, about 2.9:1, about 3:1, about 3.5:1, about 4:1, about 4.5:1, about 5:1, about 6:1, about 7:1, about 8:1, about 9:1, about 10:1, about 11:1, about 12:1, about 13:1, about 14:1, about 15:1, about 16:1, about 17:1, about 18:1, about 19:1, about 20:1, about 21:1, about 22:1, about 23:1, about 24:1, about 25:1, about 26:1, about 27:1, about 28:1, about 29:1, about 30:1, about 35:1, about 40:1, about 45:1, about 50:1, about 60:1, about 70:1, about 80:1, about 90:1, about 100:1, about 150:1, about 200:1, about 250:1, about 300:1, about 400:1, about 500:1, about 600:1, about 700:1, about 800:1, or about 900:1.

In some embodiments, the cannabinoid component includes the cannabinoid in a concentration of at least about 0.001 mg/g. In a non-limiting example, the cannabinoid component may comprise the cannabinoid in an amount of from about 0.001 mg/g to about 100 mg/g, including any amount therebetween or any ranges therein. For example, and without wishing to be limiting, the cannabinoid component may comprise the cannabinoid in an amount of from about 0.002 mg/g to about 100 mg/g, from about 0.1 mg/g to about 75 mg/g, or from about 0.1 mg/g to about 50 mg/g, including any amount therebetween or any ranges therein.

In some embodiments, the cannabinoid component includes the cannabinoid in an amount of from about 1 wt. % to about 25 wt. % (the weight percentage being of the cannabinoid relative to total weight of the cannabinoid component), including any amount therebetween or any ranges therein. For example, the cannabinoid component includes the cannabinoid in an amount of from about 2.5 wt. % to about 15 wt. %, including any amount therebetween or any ranges therein, such as from about 5 wt. % to about 7.5 wt. % or from about 7.5 wt. % to about 15 wt. %, including any amount therebetween or any ranges therein.

Cannabinoids may be obtained from any suitable source material including, but not limited to, cannabis or hemp plant material (e.g., flowers, seeds, trichomes, and kief from cannabis plants) or manufactured (for example cannabinoids produced in yeast, for example as described in WO WO2018/148848). The term “cannabis plant(s)” encompasses wild type Cannabis (including but not limited to the species Cannabis sativa, Cannabis indica and Cannabis ruderalis) and also variants thereof, including cannabis chemovars (or “strains”) that naturally contain different amounts of the individual cannabinoids.

In some embodiments, the cannabinoid is obtained from a cannabis extract (e.g., resin, wax, concentrate, and distillate).

As used herein, a “cannabis extract” refers to an extract obtained from a cannabis plant material according to any procedure known in the art; such extracts yield cannabinoids in pure or isolated form. For example, a cannabis extract may be obtained by a process including an extraction step from plant materials using for example organic solvent extraction, such as extraction with CO₂, butane, ethanol, and the like. For example, a cannabis extract may be obtained by a process including an extraction step from plant materials using for example heat decarboxylation to convert cannabinoids in their acid forms to neutral forms followed by or after CO₂ extraction (under sub-critical or super-critical conditions), providing a crude extract. The crude extract may then be “winterized,” that is, extracted with ethanol to remove lipids and waxes, as described for example in U.S. Pat. No. 7,700,368, US 2004/0049059, and US 2008/0167483, which are incorporated herein by reference. Optionally, the method for obtaining the cannabis extract may further include purification steps such as a distillation step to further purify, isolate, or crystallize one or more cannabinoids, which is referred to herein as a “distillate”; US20160346339, which is incorporated herein by reference, describes a process for extracting cannabinoids from cannabis plant material using solvent extraction followed by filtration, and evaporation of the solvent in a distiller to obtain a distillate. The distillate may be further cut with one or more terpenes. The distillate may be further purified, for example using chromatographic and other separation methods known in the art, to obtain an “isolate.” In some embodiments, pure or isolated cannabinoids, such as those provided in a cannabis extract, may be combined with water, lipids, hydrocarbons, ethanol or mixtures thereof.

The cannabinoid may be an isolated cannabinoid, such as a cannabis extract, having >75% purity (as in the case of a crude extract), or >80% purity (as in the case of a distillate), or >95% purity (as in the case of an isolate). For example, and without wishing to be limiting, the cannabinoid may have a purity such as >75%, or >80%, or >90%, or >95%, or >98%, or >98%, or >99%, or >99.5%.

In an embodiment, the cannabinoid system may include up to 10% by weight cannabinoid. In select embodiments, the cannabinoid system may include from about 0.01% by weight to about 10% by weight, more particularly from about 0.1% by weight to about 8% by weight, even more particularly from about 0.5% by weight to about 5% by weight, and even more particularly still from about 1.0% by weight to about 3% by weight of cannabinoid or cannabis-derived compound. In select embodiments, the cannabinoid system may include about 1%, about 2%, about 3%, about 4%, about 5%, about 6%, about 7%, about 8%, about 9%, about 10% by weight of cannabinoid or cannabis-derived compound. In one particularly suitable embodiment, the cannabinoid systems include about 10 mg/mL total cannabinoids.

In select embodiments, the cannabinoid component includes one or more cannabinoid distillates and isolates, and in particular, the cannabinoid component includes CBD distillates and/or isolates; THC distillates and/or isolates; or a combination of THC and CBD distillates and/or isolates. In some embodiments, the cannabinoid component includes THC distillates and/or isolates.

In select embodiments of the cannabinoid systems disclosed herein, the cannabinoids may be introduced in the form of pure cannabinoids or as a cannabis concentrate. As used herein, “pure cannabinoids” is meant to refer to a single cannabinoid or a mixture of different cannabinoids that is free of other compounds. The pure cannabinoids may be contained in solution in a diluent or other medium or may be a liquid or solid form of the pure cannabinoids absent any diluent. In an embodiment, the pure cannabinoids are synthetic or semi-synthetic cannabinoids. As used herein, “cannabis concentrate” is meant to refer a concentrated cannabinoid system of cannabinoids, such a cannabinoid extract from a plant. Non-limiting exemplary embodiments of a cannabis concentrate include a cannabinoid distillate, a cannabinoid isolate, a cannabis oil, or any other type of extract containing one or more cannabinoids.

### Other Ingredients of the Cannabinoid Component

In some embodiments, the cannabinoid component may comprise other ingredients, including but not limited to one or more carrier oils, one or more terpenes, or any combination thereof.

As used herein, the term “carrier oil” refers to any other suitable carrier oil. Non-limiting examples of carrier oils include borage oil, coconut oil, cottonseed oil, soybean oil, safflower oil, sunflower oil, castor oil, corn oil, olive oil, palm oil, peanut oil, almond oil, sesame oil, rapeseed oil, peppermint oil, poppy seed oil, canola oil, palm kernel oil, hydrogenated soybean oil, hydrogenated vegetable oils, glyceryl esters of saturated fatty acids, glyceryl behenate, glyceryl distearate, glyceryl isostearate, glyceryl laurate, glyceryl monooleate, glyceryl, monolinoleate, glyceryl palmitate, glyceryl palmitostearate, glyceryl ricinoleate, glyceryl stearate, polyglyceryl 10-oleate, polyglyceryl 3-oleate, polyglyceryl 4-oleate, polyglyceryl 10-tetralinoleate, behenic acid, medium-chain triglycerides (MCT), long chain triglycerides (LCT), and any combination thereof. Preferably, the carrier oil is MCT.

In select embodiments, the carrier oil can reduce the viscosity of the cannabinoids and/or provide other suitable properties. Further, at least in the case of solid cannabinoids (e.g., crystalline CBD), the carrier oil aids in its dissolution and allows for emulsification of the cannabinoid.

In an embodiment, a combination of carrier oils may be used in the cannabinoid systems. When more than one carrier is used, they may be used at any amount relative to the other. In an embodiment, the first carrier oil and the second carrier oil may be used at a ratio between 10:1 and 1:10 by weight to each other. In an embodiment, the two carrier oils may be used at about a ratio of 3:1, 2:1, 1:1, 1:2 or 1:3 by weight to each other. In an embodiment, the two carrier oils may be used at about a 1:1 by weight ratio to each other.

In an embodiment, the carrier oil may improve the stability of the emulsion, for example by preventing Ostwald ripening of the cannabinoid system.

In an embodiment, the carrier oil may contribute to providing rapid onset of the cannabinoid in the cannabinoid system or a beverage prepared therefrom. The carrier oil may improve the rate of absorption and/or onset of a medicinal, therapeutic and/or recreational effect of the cannabinoids. In an embodiment, the rapid onset occurs within 60 minutes, within 30 minutes, with 15 minutes, or less from administration of the cannabinoid system to a subject (e.g., in the form of a beverage). The carrier oil may also improve the rate of release of the cannabinoids into a beverage to provide an improved medicinal, therapeutic or recreational effect.

In select embodiments, the cannabinoid systems may include up to 20% by weight carrier oil. In an embodiment, the cannabinoid systems include from about 0.01% by weight to 10% by weight, more particularly from about 0.1% by weight to about 8% by weight, even more particularly from about 0.5% by weight to about 5% by weight, and even more particularly still from about 1.0% by weight to about 3% by weight carrier oil. In select embodiments, the cannabinoid system may include about 1%, about 2%, about 3%, about 4%, about 5%, about 6%, about 7%, about 8%, about 9%, about 10% by weight carrier oil.

In some embodiments, the cannabinoid component includes the carrier oil in an amount required to achieve a desired cannabinoid and/or carrier oil concentration(s), whether that be in the cannabinoid component, the nanoemulsion, the cannabinoid system, or the cannabis-infused product.

For example, the cannabinoid component may include the carrier oil in an amount of from about 1 wt. % to about 25 wt. % (the weight percentage being of the carrier oil relative to total weight of the cannabinoid component), including any amount therebetween or any ranges therein.

For example, the cannabinoid component may include the carrier oil in an amount of from about 2.5 wt. % to about 15 wt. %, including any amount therebetween or any ranges therein, such as from about 5 wt. % to about 7.5 wt. % or from about 7.5 wt. % to about 15 wt. %, including any amount therebetween or any ranges therein, based on the total weight of the cannabinoid component.

For example, the cannabinoid component may include the carrier oil in an amount such as to allow incorporating an amount of cannabinoid into the cannabinoid component of from about 0.001 mg/g to about 100 mg/g, including any amount therebetween or any ranges therein. For example, and without wishing to be limiting, the cannabinoid component may include the carrier oil in an amount such as to allow incorporating an amount of cannabinoid into the cannabinoid component of from about 0.002 mg/g to about 100 mg/g, from about 0.1 mg/g to about 75 mg/g, or from about 0.1 mg/g to about 50 mg/g, including any amount therebetween or any ranges therein.

In some embodiments, the cannabinoid component includes the carrier oil and the cannabinoid in a ratio (wt.:wt.) required to achieve the desired particle size and/or stability. For example, the cannabinoid and the carrier oil can be present in a ratio cannabinoid to carrier oil (wt.:wt.) of from about 0.40 to about 1.00, including any ratio values therein or any ratio ranges there in-between. For example, a ratio of from about 0.45 to about 0.80, or from about 0.50 to about 0.75, or from about 0.56 to about 0.71. Generally speaking, going beyond the ratio of 0.56 to 0.71 increases particle sizes.

As used herein, the term “terpenes” refers to refer to a class of chemical components comprised of the fundamental building block of isoprene, which can be linked to form linear structures or rings. Terpenes may include hemiterpenes (single isoprenoid unit), monoterpenes (two units), sesquiterpenes (three units), diterpenes (four units), sesterterpenes (five units), triterpenes (six units), and so on. At least some terpenes are expected to interact with, and potentiate the activity of, cannabinoids. Examples of terpenes known to be extractable from cannabis include aromadendrene, bergamottin, bergamotol, bisabolene, borneol, 4-3-carene, caryophyllene, cineole/eucalyptol, p-cymene, dihydroj asmone, elemene, farnesene, fenchol, geranylacetate, guaiol, humulene, isopulegol, limonene, linalool, menthone, menthol, menthofuran, myrcene, nerylacetate, neomenthylacetate, ocimene, perillylalcohol, phellandrene, pinene, pulegone, sabinene, terpinene, terpineol, 4-terpineol, terpinolene, and derivatives thereof. Additional examples of terpenes include nerolidol, phytol, geraniol, alpha-bisabolol, thymol, genipin, astragaloside, asiaticoside, camphene, beta-amyrin, thujone, citronellol, 1,8-cineole, cycloartenol, and derivatives thereof.

The reader will readily understand that in some embodiments, the cannabinoid component may include other ingredients in addition to the above described additional ingredients.

### Emulsifiers

The cannabinoid systems disclosed herein include one or more emulsifiers to stabilize the cannabinoid component described above, to reduce the particle size of the particles in the mixture and/or to provide other suitable properties. An emulsifier is a surfactant that stabilizes emulsions. Emulsifiers coat droplets within an emulsion and prevent them from coming together, or coalescing.

In some embodiments, the emulsifier is a non-ionic emulsifier. All non-ionic emulsifiers consist of a molecule that combines both hydrophilic and lipophilic groups.

In some embodiments, the emulsifier has a Hydrophilic-lipophilic balance (HLB) 10. For example, a HLB of 11, 12, 13, 14, 15, 16, 17, or 18.

As used herein, the term Hydrophilic-lipophile balance (HLB) refers to the balance of the size and strength of the hydrophilic (water-loving or polar) and the lipophilic (oil-loving or non-polar) groups of the non-ionic emulsifier. However, as used herein, HLB value is not limited to its application to non-ionic emulsifiers alone. While HLB value has a specific meaning for non-ionic emulsifiers, its meaning can be extrapolated to other emulsifiers, regardless of whether it is ionic or non-ionic, as a general indicator of the hydrophilicity and lipophilicity. Therefore, it is contemplated that other types of emulsifiers may be useful within the scope of the present disclosure. For example, suitable emulsifiers may also be selected from the group consisting of anionic, cationic and amphoteric emulsifiers.

The HLB value is also an indication of the solubility of the emulsifiers. For example, conventional understanding is that a emulsifier having a high HLB value (i.e., 8-18 according to HLB:ICI Americas Inc., “The HLB System a Time Saving Guide to Emulsifier Selection”, Chemmunique, March 1980) will tend to be water-soluble and is used when it is desired that the final product exhibit aqueous characteristics, i.e., to dilute readily with water. It is well known that an emulsifier having a low HLB value (below 9) will tend to be oil-soluble and one having a high HLB value (above 13) will tend to be water-soluble.

The HLB value for a given emulsifier is generally known by those skilled in the art or may be calculated using the Griffin's Mathematical Method (HLB=20×((Mh/M), wherein Mh=molecular weight of hydrophilic groups; M=molecular weight of the whole molecule). Alternatively, HLB values for a particular emulsifier may be determined by dividing the hydrophilic molecular weight percentage of the compound by 5.

Alternatively, the HLB values for emulsifiers may be listed in Kirk-Othmer, Encyclopedia of Chemical Technology, third edition 1979, vol. 8, page 913; and HLB:ICI Americas Inc., “The HLB System a Time Saving Guide to Emulsifier Selection”, Chemmunique, March 1980. The HLB value of an emulsifier is related to its solubility.

In some embodiments, the emulsifier includes a polysorbate-type non-ionic surfactant. For example, polyoxyethylene monostearate (PEG 400 Monostearate), polyoxyethylene monooleate (PEG 400 Monoleate), polyoxyethylene sorbitan monolaurate (Tween® 20), polyoxyethylene sorbitan monolaurate (Tween® 21), polyoxyethylene sorbitan monopalmitate (Tween® 40), polyoxyethylene sorbitan monostearate (Tween® 60), polyoxyethylene sorbitan monostearate (Tween® 61), polyoxyethylene sorbitan tristearate (Tween® 65), polyoxyethylene sorbitan monooleate (Tween® 80), polyoxyethylene sorbitan monooleate (Tween 81), polyoxyethylene sorbitan trioleate (Tween® 85), polyoxyethylene-(15)-stearic acid (Pegosperse 1500MS), polyoxyethylene-(20)-stearyl alcohol (Brij 78), polyoxyethylene-(23)-lauryl alcohol (Brij 35), (Lutensol ON 60), PEG-40 hydrogenated castor oil (Cremophor/Kolliphor RH 40), PEG-35 castor oil (Cremophor EL), Solutol HS-15 and a combination thereof. Preferably, the emulsifier includes polyoxyethylene sorbitan monooleate (Tween® 80).

The person skilled in the art would be familiar with formulating cannabinoid emulsions and would be aware of suitable amounts/ratios of the at least one emulsifier. For example, the cannabinoid systems may include up to about 10% by weight emulsifier. In an embodiment, the cannabinoid systems include from about 0.01% by weight to 10% by weight, more particularly from about 0.1% by weight to about 8% by weight, even more particularly from about 0.5% by weight to about 5% by weight, and even more particularly still from about 1.0% by weight to about 3% by weight emulsifier. In select embodiments, the cannabinoid system may include about 1%, about 2%, about 3%, about 4%, about 5%, about 6%, about 7%, about 8%, about 9%, about 10% by weight emulsifier.

In some embodiments, the cannabinoid systems of the present disclosure may include a ratio by weight of emulsifier to cannabinoid of from 10:1 to 1:10, including any ratio value therein or any range of ratio values there in-between. For example, the ratio of emulsifier to cannabinoid may be of about 3:1, about 2:1, about 1:1, about 1:2, about 1:3, and the like.

In some embodiments, the cannabinoid systems of the present disclosure may include a ratio by weight of emulsifier to carrier oil of from 5:1 to 1:1, including any ratio value therein or any range of ratio values there in-between. For example, the ratio of emulsifier to carrier oil may be of about 3:1, about 2:1, about 1:1, about 1:2, about 1:3, and the like. Preferably, the ratio of emulsifier to carrier oil is of about 2:1.

### Water Phase

The cannabinoid systems of the present disclosure comprise a cannabinoid component including a cannabinoid, and an emulsifier for use with a water phase comprising a polyhydric alcohol and a monohydric alcohol to generate a nanoemulsion.

Polyhydric alcohols (also called sugar alcohols) are water-soluble solids that can occur naturally or be produced industrially by hydrogenation of sugars. In some embodiments, the polyhydric alcohols have the general formula HOCH₂(CHOH)ₙCH₂OH. Most have five- or six-carbon chains, because they are derived from pentoses (five-carbon sugars) and hexoses (six-carbon sugars), respectively. Sugar alcohols are used widely in the food industry as thickeners and sweeteners. In commercial foodstuffs, sugar alcohols are commonly used in place of table sugar (sucrose), often in combination with high-intensity artificial sweeteners, in order to offset their low sweetness.

In some embodiments, the polyhydric alcohol is one or more of glycerol, arabitol, Hydrogenated starch hydrolysates (HSHs), lactitol, mannitol, maltitol, sorbitol, xylitol, erythritol, and isomalt. Preferably, the polyhydric alcohol is glycerol, sorbitol, or a combination thereof. More preferably, the polyhydric alcohol is glycerol.

Monohydric alcohols are hydroxy derivatives of hydrocarbons that contain a single OH group (hence the prefix “mono”). Preferably, the general formula of monohydric alcohols is CₙH₂ₙ+1OH where n is an integer selected from 2 to 8. Monohydric alcohols include at least 3 types, namely: primary alcohols where the —OH group is attached to the primary carbon atom or a —CH₂OH group is present in the molecule; secondary alcohols where the —OH group is attached to the secondary carbon atom or a >CHOH group is present in the molecule; and tertiary alcohols where the —OH group is attached to the tertiary carbon atom or a ≡COH group is present in the molecule.

In some embodiments, the monohydric alcohol is one or more of methanol, ethanol, propan-1-ol, propan-2-ol, butan-1-ol, butan-2-ol, 2-methyl propan-1-ol, or 2-methyl propan-1-ol. Preferably the monohydric alcohol is ethanol.

In some embodiments, once formed, the nanoemulsion comprises the polyhydric alcohol in an amount of about 20 wt. % or less. For example, the nanoemulsion comprises from about 10 wt. % to about 20 wt. %, including any values therein and any ranges there in-between. For example, once formed, the nanoemulsion comprises from about 12.5 wt. % to about 18.5 wt. %, from about 14.5 wt. % to about 16.5 wt. %, or about 15 wt. %.

In some embodiments, once formed, the nanoemulsion comprises the monohydric alcohol in an amount of from about 1.0 wt. % to about 10 wt. %, preferably from about 2.0 wt. % to about 8.0 wt. %, more preferably from about 2.0 to about 5.5 wt. %. Generally, going beyond a range of 2.2 wt. % to 5.4 wt. % increased particle size.

### Stabilizers

In some embodiments, the cannabinoid systems of the present disclosure may be used in combination with a stabilizer. The stabilizer may be added to the cannabinoid systems or to the cannabis-infused product.

Thus, in an embodiment, the cannabinoid systems of the present disclosure comprise a stabilizer. In an embodiment, a cannabis-infused product comprises a stabilizer. The stabilizer may be added to the product before, during or after admixture with the cannabinoid system described herein.

As used herein, a stabilizer is any substance used to prevent an unwanted change in state in the cannabinoid systems or cannabis-infused product produced therefrom (e.g., prevent degradation). The stabilizer may be used to improve or maintain the stability of the cannabinoid system itself or to improve or maintain the stability of individual components of the cannabinoid system (e.g., the cannabinoid) or of the cannabis-infused product produced therefrom. For example, the cannabinoid within the cannabinoid system or cannabis-infused product produced therefrom may be susceptible to degradation, such as oxidative degradation. Thus, in an embodiment, the stabilizer protects the cannabinoid from degradation.

Non-limiting examples of stabilizers include hydrocolloids (such as alginate, agar, carrageenan, cellulose and cellulose derivatives, gelatin, guar gum, gum Arabic, locust bean gum, pectin, starch and xanthan gum), antioxidants (water-soluble and/or oil-soluble), and chelating agents.

Water-soluble antioxidants may enhance the stability of the cannabinoid systems and/or cannabis-infused products containing the cannabinoid system by reacting with reactive species in the polar (e.g., aqueous) phase. Oil-soluble antioxidants may enhance the chemical stability of the cannabinoid system and/or cannabis-infused products containing the cannabinoid system by reacting with reactive species in the oil phase. Non-limiting examples of reactive species include peroxides, free radicals and oxygen.

Non-limiting examples of antioxidants include ascorbic acid, ascorbic acid-6 palmitate (ascorbyl palmitate), ascorbyl stearate, alpha-tocopherol, beta-carotene, butylated hydroxyaniline (BHA), butylated hydroxytolulene (BHT), delta-tocopherol, dodecyl gallate, erythorbic acid, gamma-tocopherol, glutathione, lipoic acid, octyl gallate, propyl gallate, mixed tocopherols (e.g., Fortium™), vitamin E (e.g., Tocobiol™ Plus CP60), Tocobiol™ Plus L-70, Tocobiol™ Plus GP, Tocobiol™ Plus PV, Nutrabiol T, sodium ascorbate, sodium erythorbate, and Extract of Rosemary (OxiKan™ CL).

In an embodiment, the cannabinoid systems of the present disclosure are used in combination with an antioxidant stabilizer. In an embodiment, the antioxidant stabilizer is ascorbic acid-6 palmitate (E-304) or a tocopherol.

Chelating agents may enhance the chemical stability of the cannabinoid systems and/or cannabis-infused products containing the cannabinoid system by binding dissolved metal ions. Dissolved metal ions, for example copper ions or iron ions, may catalyze oxidation-reduction reactions (redox) between dissolved oxygen and the components of the cannabinoid system or cannabis-infused product. In particular, cannabinoids may be susceptible to oxidation catalyzed by dissolved metal ions. Non-exclusive examples of chelating agents include: aminopolycarboxylic acids including ethylenediaminetetraacetic acid (EDTA) and its various salts, calixarenes, porphyrins, bipyridines, citric acid, iminodisuccinic acid, and polyaspartic acid.

In an embodiment, the cannabinoid systems of the present disclosure are used in combination with a chelating agent as a stabilizer. In an embodiment, the chelating agent is ethylenediaminetetraacetic acid (EDTA). In an embodiment, the EDTA is disodium EDTA, calcium disodium EDTA, or tetrasodium EDTA. In a particular embodiment, the EDTA is calcium disodium EDTA.

The stabilizer may be added to the cannabinoid systems, the product base or to the cannabis-infused product produced therewith. In an embodiment, the stabilizer is added to the product base separately from the cannabinoid system. In an embodiment, the stabilizer is added to the product base or to the cannabis-infused product within 30 minutes before or after the cannabinoid system is added, respectively.

In an embodiment, the stabilizer is added to the cannabinoid system. In an embodiment, the stabilizer is added to the cannabinoid system within 30 minutes before the cannabinoid system is added to the product base to make the cannabis-infused product. In an embodiment, the stabilizer is added to the cannabinoid system within 1 minute before the cannabinoid system is added to the product base to make the cannabis-infused product.

The stabilizer may be added to the cannabinoid system, the product base or to the cannabis-infused product produced therewith at any suitable concentration. In an embodiment, the stabilizer is added in a minor amount. As used herein, by “in a minor amount”, it is meant that the stabilizer is added at a concentration of between 1 ppm and 100 ppm, between 10 ppm and 50 ppm, or between 20 ppm and 30 ppm.

The use of a stabilizing agent is sometimes to the detriment of other important characteristics of a consumer product, e.g., a beverage. For example, additional components such as stabilizers may promote turbidity, cloudiness or an undesired taste profile in the final product. Also, stabilization of one component (e.g., the nanoemulsion) may have a negative effect on the stability of another component (e.g., the active ingredient). This may be particularly so for emulsification products where the development of a cannabinoid system that is clear in appearance, easy to drink (“clean” taste profile), shelf stable, and quick acting are all relevant considerations.

In an embodiment, the cannabinoid systems of the present disclosure are used in combination with a stabilizer. In an embodiment, the stabilizer is one that complements one or more components of the cannabinoid system to provide a cannabis-infused product that is clear in appearance, chemically stable, shelf stable, and/or suitable for use in a broad range of product having different characteristics (e.g., pH, high or low ionic conditions, wide array of ingredients, etc.). By “chemically stable”, it is meant that the stability of the active ingredient is improved.

In an embodiment, a chelating agent is used in combination with the cannabinoid systems of the present disclosure. In an embodiment, a chelating agent is used in combination with a cannabinoid system of the present disclosure that comprises a polyhydric alcohol and a monohydric alcohol. In an embodiment, the chelating agent is EDTA.

Without being bound by any theory, it is believed that in some embodiments, a polyhydric alcohol and a monohydric alcohol on the one hand and a chelating agent on the other hand may complement each other in that the polyhydric alcohol and a monohydric alcohol may strengthen the emulsion (e.g., stabilize the emulsion) as evidenced by improved clarity, while the chelating agent may stabilize the cannabinoids. This is an advantageous result since it was found that a chelating agent renders the aqueous product more turbid. Combined with a polyhydric alcohol and a monohydric alcohol, the clarity of the product improves.

The cannabinoid system and/or the cannabis-infused products containing the cannabinoid systems may be treated to reduce the oxygen content as this may further enhance their chemical stability. Reducing the oxygen concentration may reduce the rate of oxidative degradation and enhance the chemical stability of the cannabinoid system and/or the cannabis-infused products containing the cannabinoid system. Non-limiting examples of treatments to reduce oxygen concentration include sparging (with nitrogen and/or other inert gases), freeze-pump-thaw cycling, and treatment with reducing agents (e.g., potassium metabisulphite).

In an embodiment, cannabis-infused products made with the cannabinoid systems of the present disclosure are sparged after the cannabinoid system is added. In an embodiment, the products are sparged within at least 60 minutes, at least 45 minutes, at least 30 minutes, at least 10 minutes, at least 5 minutes or less, after the cannabinoid system is added. In an embodiment, the sparged products are also treated with reducing agents.

The cannabinoid systems and/or the cannabis-infused products containing the cannabinoid systems may be treated with agents to adjust the pH. In an embodiment, the pH of the cannabinoid system and/or the cannabis-infused products containing the cannabinoid system is adjusted to about 6.5 or less. In an embodiment, the pH of the cannabinoid system and/or the cannabis-infused products containing the cannabinoid system is adjusted to between about 2.5 and about 4.5, or between about 3.8 and about 4.3. In an embodiment, the cannabinoid system and/or cannabis-infused products containing the cannabinoid system may contain buffers to maintain a constant pH.

### Other Additives

In some embodiments, the cannabinoid systems or cannabis-infused products of the present disclosure may include any number of other additives, including without limitation a solvent, a bulking agent, an antioxidant, or a nutritional supplement. These components may be used either alone or in combination to improve, for example, the chemical and/or physical properties, stability, nutritional profile, taste, colour and/or viscosity, of the cannabinoid systems disclosed herein or a cannabis-infused beverage or foodstuff produced therefrom. In an embodiment, the antioxidant may be ascorbyl palmitate or alpha-tocopherol.

Yet other suitable types of modifiers and additives that may be used in the cannabinoid systems or cannabis-infused products disclosed herein include viscosity modifiers, natural emulsifiers, oils, thickening agents, minerals, acids, bases, vitamins, flavours, colourants, sweeteners (e.g., liquid sweeteners), and the like and combinations thereof, as known in the beverage and food arts, to provide improved solubility, stability, bioavailability, colour and taste.

Nutritional supplements comprise substances useful to the consumer of the cannabinoid systems disclosed herein, or beverages or foodstuffs prepared therewith, for maintenance of normal body health. Suitable nutritional supplements may comprise, for example, essential nutrients including vitamins, dietary minerals, amino acids and fatty acids. Exemplary nutritional supplements may include vitamin A, vitamin 1, vitamin B2, vitamin B3, vitamin B5, vitamin B6, vitamin B7, vitamin B9, vitamin B12, vitamin C, vitamin D, vitamin E, vitamin K calcium, phosphorus, potassium, sulfur, sodium, chlorine, magnesium, iron, cobalt, copper, zinc, molybdenum, iodine, selenium, manganese, nickel, chromium, fluorine, boron, strontium histidine, isoleucine, leucine, lysine, methionine, cysteine, phenylalanine, tyrosine, threonine, tryptophan, valine, alpha-linoleic acid, and linoleic acid.

Viscosity modifiers include any compound or agent capable of altering the viscosity of the cannabinoid systems disclosed herein, or a beverage or foodstuff produced therewith. Exemplary embodiments of viscosity modifiers include anticaking agents, antifoaming agents, bulking agents, coagulation agents, gelling agents, glazing agents, humectants, leavening agents, tenderizers, and thickeners. In an embodiment, the viscosity modifying agent may be an unmodified starch, pregelatinized starch, cross-linked starches, gums (e.g., guar gum, xanthum gum, acacia), polyvinyl pyrrolidone (PVP), polyethylene oxide, waxes (e.g., beeswax), and mixtures thereof.

In particularly suitable embodiments, the cannabinoid systems of the present disclosure are shelf-stable. As used herein, “shelf-stable” refers to the cannabinoid system maintaining a homogeneous mixture (i.e., no phase separation) for a period of at least 30 days, more suitably, at least 40 days, even more suitably, at least 45 days, and more suitably, at least 50 days, and even more suitably, at least 55 days or longer.

In particularly suitable embodiments, the cannabinoid systems of the present disclosure enhance or maintain the stability of the cannabinoid in the cannabinoid system, in a cannabis-infused product produced therefrom (e.g., beverage), or both. In an embodiment, loss of cannabinoid in the cannabinoid systems of the present disclosure is less than 35% by weight in 3 months, more particularly less than 25% by weight in 3 months, and more particularly still less than 20% by weight in 3 months. In an embodiment, loss of cannabinoid in the cannabinoid systems of the present disclosure is about 25%, about 20%, about 15%, about 10%, about 5%, or less, by weight in 3 months. In an embodiment, loss of cannabinoid in the cannabinoid systems of the present disclosure is less than 16% by weight THC content in 70 days.

In particularly suitable embodiments, the cannabinoid systems of the present disclosure, when mixed with an aqueous solution, provide a cannabis-infused product which is stable. By “stable”, it is meant that the cannabinoid system remains free from one or more deleterious changes over a period of time, for example at least or longer than 1 day, 1 week, 1 month, 3 months, 6 months, 1 year, or more. For example, stable may be in reference to a lack of degradation of cannabinoid; a maintenance of clarity; or a maintenance of any other property desirable for consumption.

In an embodiment, the cannabinoid system, when mixed with an aqueous solution, provides a product in which at least 60%, at least 65%, at least 70%, at least 75%, at least 80%, at least 85%, at least 90%, or at least 95% by weight of the cannabinoid remains present after about 2 months at a temperature between about 17° C. and about 40° C. In an embodiment, at least 80% by weight of the cannabinoid remains present after about 2 months at a temperature between about 17° C. and about 40° C. In an embodiment, at least 90% by weight of the cannabinoid remains present after about 2 months at a temperature between about 17° C. and about 40° C.

In an embodiment, the cannabinoid system, when mixed with an aqueous solution, provides a product in which at least 60%, at least 65%, at least 70%, at least 75%, at least 80%, at least 85%, at least 90%, or at least 95% by weight of the cannabinoid remains present after about 3 months at a temperature between about 17° C. and about 40° C. In an embodiment, at least 80% by weight of the cannabinoid remains present after about 3 months at a temperature between about 17° C. and about 40° C. In an embodiment, at least 90% by weight of the cannabinoid remains present after about 3 months at a temperature between about 17° C. and about 40° C.

In an embodiment, at least 80% by weight of the cannabinoid remains present after about 2 months at about 40° C. In an embodiment, at least 90% by weight of the cannabinoid remains present after about 2 months at about 40° C.

In an embodiment, at least 80% by weight of the cannabinoid remains present after about 3 months at about 40° C. In an embodiment, at least 90% by weight of the cannabinoid remains present after about 3 months at about 40° C.

In an embodiment, at least 84.89% by weight of the cannabinoid remains present after about 3 months at a temperature of about 40° C.

In particularly suitable embodiments, the cannabinoid systems of the present disclosure are substantially free of cyclodextrins and modified starches, thereby reducing unnatural ingredients from end use products including the cannabinoid systems.

Methods of Preparing the Cannabinoid systems

It has been found that by mixing the above-described components of the cannabinoid systems in a particular order, a concentrated nanoemulsion can be formed that, when mixed with an aqueous solution, can form a transparent or translucent nanoemulsion having favorable pharmacokinetics, for example, rapid onset, shorter duration, and minimal food effect. In select embodiments, the cannabinoid systems produced by the disclosed methods may also be clear, rather than cloudy.

The manufacturing method includes providing an oil phase and a water phase.

The oil phase includes the emulsifier and the cannabinoid component. As discussed earlier in this text, the emulsifier has a HLB 10. Surprisingly, the present inventors discovered that including the emulsifier in the oil phase rather than in the water phase allowed achieving the herein described technical advantages, such as small nanoparticle size and stability. Indeed, in a comparative example (not shown), the inventors mixed a water phase comprising 75 wt. % water, 10 wt. % Tween 80, and 10 wt. % a monohydric alcohol with an oil phase comprising 4 wt. % MCT and 1 wt. % THC (25 mg/mL THC), and this resulted in a monomodal emulsion having a particle size of >1000 nm. This was surprising because the accepted practice in the art of making nanoemulsions is that emulsifiers having a HLB 10 should be present in the water phase before mixing the oil and water phases. In the present case, it was thus counter-intuitive to introduce the emulsifier in the oil phase prior to mixing the water and oil phases.

The water phase includes the polyhydric alcohol and a monohydric alcohol. Surprisingly, the present inventors discovered that including both the polyhydric alcohol and monohydric alcohol in the water phase allowed achieving the herein described technical advantages, such as small nanoparticle size and stability. Indeed, in a comparative example (not shown), the inventors generated comparative cannabinoid emulsification systems that lack one of the polyhydric alcohol and monohydric alcohol, and this resulted in a cloudy nanoemulsion with a particle size >150 nm. This was surprising because while there have been reports of self-emulsifying nanoemulsion systems that may include a monohydric alcohol, to the inventors' knowledge, there are no reports where both the polyhydric alcohol and monohydric alcohol are required for self-emulsifying nanoemulsion systems to generate small nanoparticle size and obtain stability, as described herein.

Mixing the oil phase and the water phase requires step-wise adding (e.g., continuously, semi-continuously, drop by drop, etc.) the oil phase to the water phase with continuous stirring. Small oil droplets are spontaneously formed at the oil-water boundary as the surfactant molecules move from the oil phase to the water phase. Indeed, adding the water phase to the oil phase or adding the oil phase rapidly to the water phase did not result in low particle size emulsions.

Mixing the oil phase and the water phase may be performed under heated conditions. In an embodiment, the mixing is performed under heated conditions at a temperature of between about 40° C. and about 60° C. Better results were obtained with a temperature of between about 45° C. and about 55° C. In comparative examples (not shown) temperatures below this range generally led to the formation of polydisperse species while temperatures above this range generally led to increased particle size.

Incorporating additives into the cannabinoid systems may be desirable in certain applications. In such cases, it is possible to include one or more additives in the water phase and/or in the oil phase.

The present inventors have surprisingly discovered that when adding a pH-acidifying ingredient (e.g., ascorbic acid), it is preferable to incorporate into the cannabinoid systems after addition of the oil phase to the water phase. Indeed, in comparative examples (not shown) ascorbic acid was added to the water phase prior to adding the oil phase and a nanoemulsion having the low nanoscale size did not form.

The present inventors also observed that adding a preservative, such as tocobiol (vitamin E), to the oil phase did not impact particle size of the nanoemulsion generated.

### Cannabis-Infused Products

The present disclosure is further directed to using the cannabinoid systems to form end use cannabis-infused products, such as edibles, topical solids and non-edible liquids. The edibles can include, for example, beverages, liquids and foodstuffs.

Thus, the cannabinoid systems of the present disclosure may be used in the preparation of foodstuffs and beverages. As used herein, a beverage is any drink that may be consumed by a subject. A foodstuff is any substance suitable for consumption as a food.

The cannabinoid systems may be combined with any beverage-compatible or food-compatible ingredient. For example, cannabinoid systems of the present disclosure may be used directly in the preparation of foodstuffs and beverages, e.g., as an additive or ingredient. Powder cannabinoid systems may be used either directly, e.g., as an additive or ingredient, or indirectly e.g., by first dissolving the powder in a solvent (e.g., water) to form a liquid cannabinoid system prior to use. In some embodiments, the powder cannabinoid systems may be added to beverage or foodstuff directly. In other embodiments, the powder cannabinoid systems are diluted with a bulking agent. The pre-bulked and/or bulked powder cannabinoid systems can be packaged for individual servings (e.g., sachets/packets), packages in bulk within a single container, or a combination thereof.

Non-limiting examples of foodstuffs include baked goods (e.g., cookies, brownies, cake, pie, biscuits and pastries), candies (e.g., hard candy, soft candy, gummies, etc.), chocolates, lozenges, gum, mints, dried fruits, nuts, granola, truffles, caramels, chews, taffy, prepared meals, cooking ingredients (e.g., food additives, dry spices, honey, sugar, sweeteners, etc.), ground coffee, instant coffee and tea leaves.

Non-limiting examples of comprises water, oil, alcohol; with or without additives or modifiers or both. Such beverage base can be divided into various groups such as plain water, drinking water, milk (both diary and non-diary), juice, a smoothie, coffee or a caffeinated beverage, tea, herbal tea, a cocoa beverage, a carbonated drink, a nitrogenated drink, an energy drink, a drinkable yogurt, a fermented beverage, or an alcoholic or non-alcoholic drink or other hot, room temperature or cold liquids used in drinks. An alcoholic or non-alcoholic drink includes but is not limited to, beer, lager, cider, spirits, wine/fortified wine, and cocktails. Beverages can be caffeinated or non-caffeinated and may contain calories or not. Such beverages may be produced in ready to use form or be produced in a form suitable for preparation in final consumable form at or proximate to the time of ingestion.

Beverages may be packaged as individual packages, suitably single use packages, and multiple packages. The packaging can be in airtight containers. Packaging may be comprised of paper, plastic, metal, and glass. Beverages may include bubble containing or producing liquids with dissolved gas or liquids capable of producing gas proximately in time of consumption. In one embodiment of the disclosure, the beverages, optionally comprising additives, modifiers or both, are convenient to consumers, and are manufactured at modest expense. Beverages with dissolved gas may be created by a method comprising addition of carbon dioxide, ozone, oxygen, and nitrogen. For beverages with dissolved gas, dissolved gas may be added to the beverage by methods comprising application of pressure and adding water with the dissolved gas. The dissolved gas is released from the beverage when pressure is reduced as effervescence.

When making cannabis-infused beverages, the cannabinoid systems of the present disclosure typically are admixed or infused into a beverage base to produce the cannabis-infused beverage. Generally, beverage bases are liquids meeting the common meaning of the term “biocompatible”, which include materials that are not harmful to living tissue. Typically, beverage bases will make up between about 50% and 99.99% by weight or by volume of the beverage. In an embodiment, the beverage base will make up between about 80% and about 99.99% by weight of the beverage. In an embodiment, the beverage base will make up between about 80% and about 99.9% by weight of the beverage. In an embodiment, the beverage base will make up between about 95% and about 99.9% by weight of the beverage. In an embodiment, the beverage base will make up about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or more by weight of the beverage. In an embodiment, the beverage base will make up about 99.0%, about 99.1%, about 99.2%, about 99.3%, about 99.4%, about 99.5%, about 99.6%, about 99.7%, about 99.8%, or about 99.9% by weight of the beverage. In an embodiment, the beverage base is water. In an embodiment, additives may be present in addition to the quantity of beverage base. In an embodiment, a liquid additive (e.g., sweetener) may be present in addition to the quantity of beverage base.

In a particular embodiment, the beverage base will make up between about 80% and about 95% by weight of the beverage, and a liquid additive (e.g., sweetener) will make up between about 4.9% and about 14.9% by weight of the beverage. In select embodiments, the beverage base will make up about 80%, about 81%, about 82%, about 83%, about 84%, about 85%, about 86%, about 87%, about 88%, about 89%, about 90%, about 91%, about 92%, about 93%, about 94%, about 95% by weight of the beverage. In select embodiments, a liquid additive (e.g., sweetener) will make up about 18%, about 17%, about 16%, about 15%, about 14%, about 13%, about 12%, about 11%, about 10%, about 9%, about 8%, about 7%, about 6%, about 5%, about 4%, about 3% or less by weight of the beverage. In an embodiment, the beverage base is water and the liquid additive is a liquid sweetener, such as for example a 67.5° Bx (Brix) sugar solution.

The amount of the cannabinoid system of the present disclosure added to beverage bases or foodstuff bases will vary depending on the desired dosage of cannabinoid. For example, in some embodiments each serving, unit or item of foodstuff or beverage will contain about 0.5 mg to about 100 mg of cannabinoids. In an embodiment, the foodstuff or beverage will contain about 2.0 mg to about 10 mg of cannabinoids. In an embodiment, the foodstuff or beverage will contain about 0.5 mg, about 1.0 mg, about 1.5 mg, about 2.0 mg, about 2.5 mg, about 3.0 mg, about 3.5 mg, about 4.0 mg, about 4.5 mg, about 5.0 mg, about 5.5 mg, about 6.0 mg, about 6.5 mg, about 7.0 mg, about 7.5 mg, about 8.0 mg, about 8.5 mg, about 9.0 mg, about 9.5 mg, or about 10.0 mg of cannabinoids. In an embodiment, the cannabinoid is THC. In an embodiment, the cannabinoid is CBD. In an embodiment, the cannabinoid is THC and CBD, in similar or different quantities.

In an embodiment, the cannabis-infused product may comprise between about 0.5% and about 25% by weight of the cannabinoid system, more particularly between about 1% and about 10% by weight of the cannabinoid system, and more particularly still between about 1% and about 5% by weight of the cannabinoid system. In an embodiment, the product may comprise between about 0.5% and about 3% by weight of the cannabinoid system. In some embodiments, low quantities by weight of the cannabinoid system may be used due to advantageous properties of the cannabinoid systems of the present disclosure in stably formulating cannabinoids.

In another embodiment, the cannabinoid systems are absorbed into a solid material for use as an end use product. By way of example, the cannabinoid systems may be absorbed onto one or more of blotter paper, tea leaves, coffee grounds, spices and the like to allow for a convenient water-soluble edible or tea bag.

In some embodiments, the cannabinoid systems, beverages and/or foodstuffs disclosed herein provide a desired intoxication effect as measured by a standard British unit of alcohol. As used herein, “one British unit of alcohol” is defined as 10 mL (8 g) of pure alcohol. For example, the number of units of alcohol in a drink can be determined by multiplying the volume of the drink (in milliliters) by its percentage alcohol by volume (ABV), and dividing by 1000.

Suitably, in some aspects, the cannabis-infused products are formed and administered to provide a subjective or objective intoxicating effect equivalent to a standard British unit of alcohol. More particularly, from about 25 mL to 500 mL of the beverage, more particularly, from about 35 ml to about 250 ml, and even more particularly, from about 60 ml to about 120 ml of the beverage, are formed and administered to provide an intoxicating effect equivalent to a standard British unit of alcohol. By further way of example, in one aspect, consuming about 35 mL to about 60 mL of the beverage causes either a subjective or objective intoxicating effect equivalent to a standard British unit of alcohol. In another aspect, consuming about 60 mL to about 120 mL of the beverage causes either a subjective or objective intoxicating effect equivalent to a standard British unit of alcohol. In yet another aspect, consuming about 120 mL to about 250 mL of the beverage causes either a subjective or objective intoxicating effect equivalent to a standard British unit of alcohol. In yet another aspect, consuming about 250 mL to about 500 mL of the beverage causes either a subjective or objective intoxicating effect equivalent to a standard British unit of alcohol.

It will further be appreciated that in certain embodiments the cannabis-infused products should provide the human or non-human subject an intoxicating effect at the desired time. For example, in some embodiments, the beverage or foodstuff provides for an onset of intoxication in a time period of from about 10 minutes to about 120 minutes, including from about 20 minutes to about 90 minutes, and including from about 30 minutes to about 60 minutes, after consumption of the beverage or foodstuff. By way of further example, in certain embodiments the beverage or foodstuff can be formed and administered to provide for an onset of the intoxication of about 10 minutes, or about 15 minutes, or about 20 minutes, or about 25 minutes, or about 30 minutes, 40 minutes, 60 minutes, 90 minutes, or even 120 minutes. In further examples and embodiments, the beverage or foodstuff can be formed and administered to provide for an onset of the intoxication of about 180 minutes, or even about 240 minutes, or even still about 300 minutes.

Advantageously, embodiments of the cannabis-infused products comprising or produced using the cannabinoid systems of the present disclosure are shelf-stable.

As used in the context of the products herein, “shelf-stable” refers to the cannabinoid system maintaining its water-soluble nature in an aqueous product at least in respect of the cannabinoid (e.g., no precipitation of these compounds) for a period of at least 30 days, more suitably, at least 40 days, even more suitably, at least 45 days, and more suitably, at least 50 days, and even more suitably, at least 55 days or longer.

In particularly suitable embodiments, the cannabis-infused products disclosed herein enhance or maintain the stability of the cannabinoids. In an embodiment, loss of cannabinoids in the cannabis-infused products disclosed herein is less than 35% by weight in 3 months, more particularly less than 25% by weight in 3 months, and more particularly still less than 20% by weight in 3 months. In an embodiment, loss of cannabinoids in the cannabis-infused products disclosed herein is about 25%, about 20%, about 15%, about 10%, about 5%, or less, by weight in 3 months. In an embodiment, loss of cannabinoids in the cannabis-infused products disclosed herein is less than 16% by weight THC content in 70 days.

In particularly suitable embodiments, the cannabis-infused products disclosed herein are stable. By “stable”, it is meant that the products remain free from one or more deleterious changes over a period of time, for example at least or longer than 1 day, 1 week, 1 month, 3 months, 6 months, 1 year, or more. For example, stable may be in reference to a lack of degradation of cannabinoids; a maintenance of clarity; or a maintenance of any other property desirable for consumption.

In an embodiment, at least 60%, at least 65%, at least 70%, at least 75%, at least 80%, at least 85%, at least 90%, or at least 95% by weight of the original or time 0 quantity of the cannabinoid remains present in the product after about 2 months at a temperature between about 17° C. and about 40° C. In an embodiment, at least 80% by weight of the original or time 0 quantity of the cannabinoid remains present in the product after about 2 months at a temperature between about 17° C. and about 40° C. In an embodiment, at least 90% by weight of the original or time 0 quantity of the cannabinoid remains present in the product after about 2 months at a temperature between about 17° C. and about 40° C.

In an embodiment, at least 60%, at least 65%, at least 70%, at least 75%, at least 80%, at least 85%, at least 90%, or at least 95% by weight of the original or time 0 quantity of the cannabinoid remains present in the product after about 3 months at a temperature between about 17° C. and about 40° C. In an embodiment, at least 80% by weight of the original or time 0 quantity of the cannabinoid remains present in the product after about 3 months at a temperature between about 17° C. and about 40° C. In an embodiment, at least 90% by weight of the original or time 0 quantity of the cannabinoid remains present in the product after about 3 months at a temperature between about 17° C. and about 40° C.

In an embodiment, at least 60%, at least 65%, at least 70%, at least 75%, at least 80%, at least 85%, at least 90%, or at least 95% by weight of the original or time 0 quantity of the cannabinoid remains present in the product after about 6 months at a temperature between about 17° C. and about 40° C. In an embodiment, at least 80% by weight of the original or time 0 quantity of the cannabinoid remains present in the product after about 6 months at a temperature between about 17° C. and about 40° C. In an embodiment, at least 90% by weight of the original or time 0 quantity of the cannabinoid remains present in the product after about 6 months at a temperature between about 17° C. and about 40° C.

In an embodiment, at least 80% by weight of the original or time 0 quantity of the cannabinoid remains present in the product after about 2 months at about 40° C. In an embodiment, at least 90% by weight of the original or time 0 quantity of the cannabinoid remains present in the product after about 2 months at about 40° C.

In an embodiment, at least 80% by weight of the original or time 0 quantity of the cannabinoid remains present in the product after about 3 months at about 40° C. In an embodiment, at least 90% by weight of the original or time 0 quantity of the cannabinoid remains present in the product after about 3 months at about 40° C.

In an embodiment, at least 80% by weight of the original or time 0 quantity of the cannabinoid remains present in the product after about 6 months at about 40° C. In an embodiment, at least 90% by weight of the original or time 0 quantity of the cannabinoid remains present in the product after about 6 months at about 40° C.

In any of the embodiments described herein, the cannabis-infused products may have a reduced oxygen content, such as by removing the oxygen by means of equipment designed to perform this function or by chemical removal (e.g., N₂ purge and/or potassium disulfite). In an embodiment, the oxygen content of the product is between about 0 ppm and about 500 ppm. In an embodiment, the product is sealed until use in order to maintain the reduced oxygen content.

### Uses of Cannabis-Infused Products

Cannabis-infused products are typically used for recreational and/or medicinal uses. For example, cannabis-infused products can be used to achieve a desired effect in a user, such as a psychoactive effect, a physiological effect, or a treatment of a condition. By “psychoactive effect”, it is meant a substantial effect on mood, perception, consciousness, cognition, or behavior of a subject resulting from changes in the normal functioning of the nervous system. By “physiological effect”, it is meant an effect associated with a feeling of physical and/or emotional satisfaction. By “treatment of a condition”, it is meant the treatment or alleviation of a disease or condition by absorption of cannabinoid(s) at sufficient amounts to mediate the therapeutic effects.

The terms “treating”, “treatment” and the like are used herein to mean obtaining a desired pharmacologic and/or physiologic effect. The effect may be prophylactic, in terms of completely or partially preventing a disease, condition, or symptoms thereof, and/or may be therapeutic in terms of a partial or complete cure for a disease or condition and/or adverse effect, such as a symptom, attributable to the disease or disorder. “Treatment” as used herein covers any treatment of a disease or condition of a mammal, such as a dog, cat or human, preferably a human.

In certain embodiments, the disease or condition is selected from the group consisting of pain, anxiety, an inflammatory disorder, a neurological disorder, a psychiatric disorder, a malignancy, an immune disorder, a metabolic disorder, a nutritional deficiency, an infectious disease, a gastrointestinal disorder, and a cardiovascular disorder. Preferably the disease or condition is pain. In other embodiments, the disease or condition is associated with the feeling of physical and/or emotional satisfaction.

In the context of recreational use, the “effective amount” administered and rate and time-course of administration, will depend on the desired effect associated with a feeling of physical and/or emotional satisfaction in the subject.

In the context of health and wellness use, the “effective amount” administered and rate and time-course of administration will depend on the nature and severity of the disease or condition being treated and typically also takes into consideration the condition of the individual subject, the method of administration and the like.

### EXAMPLES

The following examples are included to demonstrate various embodiments of the present disclosure. It should be appreciated by those of skill in the art that the techniques disclosed in the examples that follow represent techniques discovered by the inventors to function well in the practice of the present disclosure, and thus may be considered to constitute preferred modes for its practice. However, those of skill in the art should, in light of the present disclosure, appreciate that many changes can be made in the specific embodiments which are disclosed and still obtain a like or similar result without departing from the scope of the present disclosure.

### Example 1

In this example, a self-emulsifying cannabinoid system in accordance with an embodiment of the present disclosure was prepared.

A water phase was provided. The water phase was made by mixing water, glycerol, a monohydric alcohol and EDTA with gentle magnetic stirring at 50° C.

An oil phase was provided. The oil phase was made by mixing Tween 80, MCT, THC distillate and tocobiol with gentle magnetic stirring at 50° C. The oil phase was added dropwise to the water phase with magnetic stirring (600 rpm) at 50° C. to generate a self-emulsifying nanoemulsion. The ingredients are set out in Table 1.

TABLE 1

| | | Percentage |
|---|---|---|
| Ingredient | Amount (g) | (wt. %) |
| Water | 124 | 62.05 |
| Glycerol | 28.89 | 14.45 |
| Ethanol | 7.48 | 3.74 |
| EDTA | 0.02 | 0.01 |
| Tween 80 | 25.66 | 12.83 |
| MCT | 7.48 | 3.74 |
| THC distillate | 5.35 | 2.68 |
| Tocobiol | 0.99 | 0.50 |

Another self-emulsifying cannabinoid system was generated in which there was no Tocobiol added. Both self-emulsifying cannabinoid systems generated a nanoemulsion having a size below 120 nm.

### Example 2

In this example, the nanoemulsion generated in Example 1 was used for infusing beverage bases and the resulting cannabis-infused beverages were tested for stability.

A dilution was prepared from the concentrated nanoemulsion (23.66 mg/mL THC) to a concentration of 0.30 mg/mL THC. All samples were filled to volume capacity of the container (glass vials filled to 15 mL and aluminum cans filled to 40 mL). All sample solutions had their headspace purged using argon gas in their final containers prior to sealing. All samples were pasteurized at 140F for 10 minutes. The beverage bases used in this example are set out in the following Table 2.

TABLE 2

| Beverage | |
|---|---|
| base | Beverage base Ingredients |
| 1 | Water, Sugars, Citric acid, Natural colorants, Natural flavour |
| 2 | Water, Sugars, Citric acid, Natural flavour. |
| 3 | Carbonated water, starch source, Sugars, Calcium chloride, |
| | Lactic acid, Calcium sulphate, Salt, Phosphoric acid, |
| | Natural flavour. |
| 4 | Carbonated water, Sugars, Citric acid, Natural colorant, |
| | Natural flavour. |
| 5 | Water |

Qualitative stability assessments were made over a period of 6 months. All cannabis-infused beverages remained stable with no visually perceivable changes in color and clarity. Quantitative assessments were also made over a period of 6 months. The quantitative tests included THC concentration (mg/mL) as determined with HPLC and nanoemulsion Particle size (PS) (nm) and poly dispersity index (P) as determined by LS). The quantitative results are reproduced in the following Table 3.

TABLE 3

| | Initial | | 1 month | | 6 months | |
|---|---|---|---|---|---|---|
| Beverage, temperature | PS | [THC] | PS | [THC] | PS | [THC] |
| and container material | (PDI) | mg/mL | (PDI) | mg/mL | (PDI) | mg/mL |
| Beverage 1, 25 C., Glass | 63 (4) | 0.285 | 61 (10) | 0.282 | 66 (14) | 0.248 |
| Beverage 1, 40 C., Glass | // | // | 66 (11) | 0.263 | 68 (6) | 0.194 |
| Beverage 1, 25 C., Can | // | // | 59 (10) | 0.281 | 66 (1) | 0.262 |
| Beverage 1, 40 C., Can | // | // | 62 (11) | 0.274 | 72 (24) | 0.232 |
| Beverage 2, 25 C., Glass | 59 (10) | 0.289 | 57 (13) | 0.269 | 59 (17) | 0.202 |
| Beverage 2, 40 C., Glass | // | // | 56 (14) | 0.206 | 62 (9) | 0.045 |
| Beverage 3, 25 C., Glass | 59 (8) | 0.296 | 57 (11) | 0.287 | 60 (12) | 0.243 |
| Beverage 3, 40 C., Glass | // | // | 58 (7) | 0.268 | 59 (12) | 0.178 |
| Beverage 4, 25 C., Glass | 65 (15) | 0.302 | 75 (26) | 0.287 | 88 (24) | 0.255 |
| Beverage 4, 40 C., Glass | // | // | 80 (25) | 0.272 | 89 (25) | 0.183 |
| Beverage 5, 25 C., Glass | 54 (4) | 0.288 | 53 (4) | 0.272 | 55 (9) | 0.208 |
| Beverage 5, 40 C., Glass | // | // | 50 (11) | 0.223 | 47 (17) | 0.092 |

These results show that the cannabis-infused beverages remained stable over time in terms of color and clarity. The cannabinoid system also remained stable over time in terms of nanoemulsion particle size and cannabinoid content.

### Example 3

In this example, a self-emulsifying cannabinoid system in accordance with an embodiment of the present disclosure was prepared as per Example 1. The ingredients are set out in Table 4.

TABLE 4

| | | Percentage |
|---|---|---|
| Ingredient | Amount (g) | (wt. %) |
| Water | 58.00 | 62.29 |
| Glycerol | 13.50 | 14.50 |
| Ethanol | 3.00 | 3.22 |
| Tween 80 | 12.36 | 13.27 |
| THC distillate | 2.50 | 2.68 |
| MCT | 3.50 | 3.76 |
| Tocobiol | 0.25 | 0.27 |

The self-emulsifying cannabinoid system generated a nanoemulsion having a size below 120 nm.

### Comparative Example 1

In this example, a commercially available comparative cannabinoid system was tested for stability in various beverage bases set out in Table 2.

The comparative cannabinoid system was used to generate a cannabinoid nanoemulsion having a size of about 40 nm and diluted into the beverage bases at a concentration of 0.03 mg/mL. Samples were stored in pasteurized aluminum cans at 25° C. or 40° C. Samples were stored for approximately 3-4 months or for 1 year. Particle size (PS) (nm) and polydispersity index (PDI %) were determined by DLS.

TABLE 5
1 year 25° C.

Beverage

| | 1 | 2 | 3 | 5 |
|---|---|---|---|---|
| PS | 165 | 48(36%) | 51(47%) | 34(37%) |
| (nm) | | 269(64%)* | 275(53%)* | 213(63%)* |
| PDI % | 20 | 22 | 28 | 28 |

TABLE 6
1 year 40° C.

Beverage

| | 1 | 2 | 3 | 5 |
|---|---|---|---|---|
| PS | 191 | 94(60%) | 35(18%) | 47(45%) |
| (nm) | | 36(40%)* | 148(82%)* | 196(55%)* |
| PDI % | 17% | 27% | 28% | 27% |

TABLE 7
3-4 months RT

Beverage

| | 2 | 4 |
|---|---|---|
| PS | 22(22%) | 207 |
| (nm) | 180(78%)* | |
| PDI % | 33% | 28% |

*A PS described with 2 values denotes a bimodal particle size distribution. The comparative cannabinoid system that was used for these samples does not conserve the initial nanoparticle 40 nm and instead migrates towards larger particle sizes with a large distribution, mostly with two distributions (bimodal).

Other examples of implementations will become apparent to the reader in view of the teachings of the present description and as such, will not be further described here.

Note that titles or subtitles may be used throughout the present disclosure for convenience of a reader, but in no way these should limit the scope of the invention. Moreover, certain theories may be proposed and disclosed herein; however, in no way they, whether they are right or wrong, should limit the scope of the invention so long as the invention is practiced according to the present disclosure without regard for any particular theory or scheme of action.

All references cited throughout the specification are hereby incorporated by reference in their entirety for all purposes.

Reference throughout the specification to “some embodiments”, and so forth, means that a particular element (e.g., feature, structure, and/or characteristic) described in connection with the invention is included in at least one embodiment described herein, and may or may not be present in other embodiments. In addition, it is to be understood that the described inventive features may be combined in any suitable manner in the various embodiments.

It will be understood by those of skill in the art that throughout the present specification, the term “a” used before a term encompasses embodiments containing one or more to what the term refers. It will also be understood by those of skill in the art that throughout the present specification, the term “comprising”, which is synonymous with “including,” “containing,” or “characterized by,” is inclusive or open-ended and does not exclude additional, un-recited elements or method steps.

Unless otherwise defined, all technical and scientific terms used herein have the same meaning as commonly understood by one of ordinary skill in the art to which this invention pertains. In the case of conflict, the present document, including definitions will control.

As used herein, the terms “preferred”, “preferably” and variants refer to embodiments of the disclosure that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the disclosure.

As used in the present disclosure, the terms “around”, “about” or “approximately” shall generally mean within the error margin generally accepted in the art. Hence, numerical quantities given herein generally include such error margin such that the terms “around”, “about” or “approximately” can be inferred if not expressly stated.

Although various embodiments of the disclosure have been described and illustrated, it will be apparent to those skilled in the art considering the present description that numerous modifications and variations can be made. The scope of the invention is defined more particularly in the appended claims.

1.-16. (canceled)

17. A self-emulsification cannabinoid system comprising:
a water phase comprising a polyhydric alcohol and a monohydric alcohol; and
an oil phase comprising an emulsifier and a cannabinoid component, the cannabinoid component including a cannabinoid,
wherein the water phase and oil phase combine to generate a nanoemulsion.

18. The system of claim 17, wherein the polyhydric alcohol is one or more of glycerol, arabitol, hydrogenated starch hydrolysates (HSHs), lactitol, mannitol, maltitol, sorbitol, xylitol, erythritol, and isomalt, preferably glycerol, sorbitol, or a combination thereof, more preferably the polyhydric alcohol is glycerol and the polyhydric alcohol is in an amount of from about 10 wt. % to about 20 wt. %, preferably from about 12.5 wt. % to about 18.5 wt. %, more preferably from about 14.5 wt. % to about 16.5 wt. %, or more preferably about 15 wt. %.

19. (canceled)

20. The system of claim 17, wherein the monohydric alcohol is in an amount of from about 1.0 wt. % to about 10 wt. %, preferably from about 2.0 wt. % to about 8.0 wt. %, more preferably from about 2.0 to about 5.5 wt. % and wherein the monohydric alcohol includes methanol, ethanol, propan-1-ol, propan-2-ol, butan-1-ol, butan-2-ol, 2-methyl propan-1-ol, or 2-methyl propan-1-ol, preferably ethanol.

21.-22. (canceled)

23. The system of claim 17, wherein the cannabinoid component further comprises a carrier oil, selected from borage oil, coconut oil, cottonseed oil, soybean oil, safflower oil, sunflower oil, castor oil, corn oil, olive oil, palm oil, peanut oil, almond oil, sesame oil, rapeseed oil, peppermint oil, poppy seed oil, canola oil, palm kernel oil, hydrogenated soybean oil, hydrogenated vegetable oils, glyceryl esters of saturated fatty acids, glyceryl behenate, glyceryl distearate, glyceryl isostearate, glyceryl laurate, glyceryl monooleate, glyceryl, monolinoleate, glyceryl palmitate, glyceryl palmitostearate, glyceryl ricinoleate, glyceryl stearate, polyglyceryl 10-oleate, polyglyceryl 3-oleate, polyglyceryl 4-oleate, polyglyceryl 10-tetralinoleate, behenic acid, medium-chain triglycerides (MCT), long chain triglycerides (LCT), and any combination thereof.

24.-25. (canceled)

26. The system of claim 23, wherein the cannabinoid and the carrier oil are present in a ratio by weight of from about 0.40 to about 1.00, preferably from about 0.45 to about 0.80, more preferably from about 0.50 to about 0.75, even more preferably from about 0.56 to about 0.71.

27. The system of claim 17, wherein the emulsifier has a Hydrophilic-lipophilic balance (HLB)≥10.

28. The system of claim 27, wherein the emulsifier includes a non-ionic emulsifier selected from polyoxyethylene monostearate, polyoxyethylene monooleate, polyoxyethylene sorbitan monolaurate, polyoxyethylene sorbitan monolaurate, polyoxyethylene sorbitan monopalmitate, polyoxyethylene sorbitan monostearate, polyoxyethylene sorbitan monostearate, polyoxyethylene sorbitan tristearate, polyoxyethylene sorbitan monooleate, polyoxyethylene sorbitan monooleate, polyoxyethylene sorbitan trioleate, polyoxyethylene-(15)-stearic acid, polyoxyethylene-(20)-stearyl alcohol, polyoxyethylene-(23)-lauryl alcohol, PEG-40 hydrogenated castor oil, PEG-35 castor oil, Solutol HS-15, and any combinations thereof, more preferably the emulsifier includes a non-ionic polysorbate surfactant, even more preferably the emulsifier includes polysorbate 80.

29. (canceled)

30. The system of claim 17, wherein the cannabinoid includes THC, CBD, CBG, CBN, or any combinations thereof.

31. The system of claim 17, wherein the nanoemulsion comprises a cannabinoid content of about 0.1 mg/ml or more.

32. The system of claim 17, wherein the nanoemulsion has a particle size of about 120 nm or less, preferably of about 100 nm or less, more preferably of about 80 nm or less, even more preferably of about 50 nm or less, even yet more preferably of about 35 nm or less, even more particularly of from about 25 nm to about 35 nm.

33. A process for manufacturing a cannabinoid nanoemulsion, comprising:
a) providing a water phase comprising a polyhydric alcohol and a monohydric alcohol;
b) providing an oil phase comprising an emulsifier and a cannabinoid component, the cannabinoid component including a cannabinoid; and
c) adding the oil phase to the water phase in a stepwise manner while mixing to generate the cannabinoid nanoemulsion.

34. The process of claim 33, wherein the stepwise manner comprises adding the oil phase dropwise to the water phase.

35. The process of claim 33, wherein the mixing is performed with stirring at about 800 rpm or less, preferably at about 600 rpm or less and at a temperature of between about 45° C. and about 55° C.

36. (canceled)

37. The process of claim 33, further comprising
d) adjusting a pH of the nanoemulsion to about 6.5 or less, preferably to between about 2.5 and about 4.5, more preferably between about 3.8 and about 4.3.

38. The process of claim 36, wherein adjusting the pH comprises adding ascorbic acid for adjusting the pH.

39.-70. (canceled)

71. A cannabis-infused product comprising the cannabinoid self-emulsification system of claim 1.

72. The cannabis-infused product of claim 71, which is a cannabis-infused edible.

73. The cannabis-infused product of claim 0, which is a cannabis-infused beverage and wherein the monohydric alcohol is ethanol.

74. The cannabis-infused product of claim 73, wherein the cannabis-infused beverage is drinking water, an energy drink, a fermented beverage, or a non-alcoholic beer, lager, cider, spirit, wine, or cocktail.

75. The cannabis-infused product of claim 71, which is a capsule.
